(19) United States
(12) Patent Application Publication
(10) Pub. No.: US 2017/0020573 A1
(43) Pub. Date: Jan. 26, 2017

(54) PEDICLE SCREW TULIP ASSEMBLY WITH MULTI-SEGMENTED MEMBER

(71) Applicant: AMENDIA, INC., Marietta, GA (US)

(72) Inventors: David Brett Cain, Marietta, GA (US); Joshua David Gunn, Woodstock, GA (US)

(73) Assignee: AMENDIA, INC., Marietta, GA (US)

(21) Appl. No.: 14/821,151

(22) Filed: Aug. 7, 2015

Related U.S. Application Data
(60) Provisional application No. 62/194,601, filed on Jul. 20, 2015.

(51) Int. Cl. A61B 17/70; A61B 17/86

(52) U.S. Cl. CPC A61B 17/7037; A61B 17/8605; A61B 17/8685; A61B 17/7002; A61B 17/7032

(57) ABSTRACT

A pedicle screw assembly has a bone screw, a tulip, a multi-segmented locking member and a saddle. The multi-segmented locking member is internal of the tulip positioned in a recess or undercut groove of an inner surface of the tulip. The saddle has a proximal end for engaging a rod and a distal end for receiving a head of the bone screw. The saddle has an exterior surface positioned between the ends. The outer surface is sized to move axially inside the tulip.

### TECHNICAL FIELD

The present invention relates to an improved pedicle screw tulip assembly.

### BACKGROUND OF THE INVENTION

Bone anchor screws come in a variety of shapes and sizes. One of the more common styles has a polyaxial head that allows for the screw to enter the bone structure at an ideal or preferred inclination. To achieve this polyaxial inclination, the head has a shape configured to rotate about its lower external surface. This lower surface can be one of a number of shapes like conical or spherical or hemispherical. This ability is often used in devices having a pedicle screw and tulip assembly.

The pedicle screw and tulip assembly generally includes a tulip. A tulip is a body structure having two opposing sides spaced by a slotted opening to receive a spinal rod. The tulip often employs internal threads to receive a rod locking set screw to anchor or fix the rod in the tulip. The lower portion of the tulip has an opening to allow the threaded shank to pass and to receive and hold the pedicle screw head in a base seat. Often, the tulip can have a saddle that supports the rod along an underside of the rod. The saddle typically has an upper recessed curvature into which the rod sits and a lower cup like opening to receive the top of the pedicle screw head. When the saddle and rod and set screw are tightened, the screw angle is fixed against the tulip seat.

In some cases, it is preferred that the pedicle screw is first placed securely in the bone structure leaving the head protruding above the bone surface. In this surgical procedure a modular head tulip assembly must be adapted to fit down onto the projecting screw head. To accomplish this, the surgeon must push the tulip onto and over the screw head without a clear path of vision. Accordingly, the placement must be accomplished without any way of knowing if the tulip or other device is properly secured. Thereafter, the device is tightened to complete the assembly and the only way to insure the assembly is secure requires an upward pulling of the tightened assembly. This is not a good test, as the assembly will be loosened or the screw to bone interface weakened.

More typically, the pedicle screw and tulip come as a pre-assembly with a pedicle screw of a given or particular size thread.

In many cases, the underlying bone structure being screwed into is insufficient in structure to work well with a normal sized pedicle screw. Ideally, a surgeon would like to use a larger sized screw to improve the bone fastening. The prior art tulips unfortunately cannot accommodate larger screws. The present invention permits this in a uniquely novel design. This allows a single tulip assembly design to accommodate a variety of sizes of pedicle screws.

These and other objectives are achieved by the invention as described hereinafter.

### SUMMARY OF THE INVENTION

The purpose of this invention is to allow a larger diameter screw to be used with a normal sized pedicle tulip through the use of a multi-segmented member placed into a groove inside the tulip to retain the pedicle screw.

Existing technology uses a single split ring or collet to retain the pedicle screw.

In the present invention, the pedicle screw head can be loaded into the bottom of the tulip until it is pushed past an undercut groove or recess inside the tulip. Then segments or pieces of a multi-segmented member are inserted, preferably, from the bottom of the tulip into the recess or groove to form a segmented locking feature. The pedicle screw head is then pulled against the multi-segmented member that is formed from the multiple segments or pieces that were inserted and then the saddle is placed on top of the screw head preventing the segment pieces from being able to come out of the recess or undercut groove. The assembly acts as any normal pedicle screw, however, with a larger screw size if desired.

The undercut groove could be made only partially around the circumference allowing fewer segment pieces to be used. The concept works well with two, three or four segments to form the locking feature.

By using multiple segment pieces instead of a split ring, allows for thicker segmented pieces to be placed in a bottom loading tulip. Normally the ring is limited by the elasticity of the material and the moment of inertia of the section allowing the ring to spring back. With the multi-locking member made in segments it has to undergo no bending stress unlike a normal split ring. This allows for a more robust lock to be installed in the groove as the segment pieces can move expanding in the small gap between segments substantially narrowing or closing the gap circumferentially as the segments are inserted into the groove below the screw head, the segment pieces retain the screw head securely on assembly. The segment pieces can be arcuate segments, straight pieces or “V” shaped pieces any of which can be loaded into the recessed opening or groove to hold the pedicle screw.

A pedicle screw assembly has a bone screw, a tulip, a multi-segmented locking member and a saddle. The multi-segmented locking member is internal of the tulip positioned in a recess or undercut groove of an inner surface of the tulip. The saddle has a proximal end for engaging a rod and a distal end for receiving a head of the bone screw. The saddle has an exterior surface positioned between the ends. The outer surface is sized to move axially inside the tulip.

The recess or undercut groove of the tulip can have a conical surface tapering inward distally or can be rounded or straight. The effective inner diameter or distance of the segment pieces of the multi-segmented locking member is defined as the distance spanning across the opening or recessed groove between segments. The tulip allows the pedicle screw head to enter through the distal opening and then, when past the recess or groove, the segment pieces can be installed to secure the screw in the tulip.

The bone screw has one of the following head shapes; at least partially a hemispherical or spherical head, conical or a radial array or loci of cylindrical surfaces or any other bulbous head. The head has a driving feature for torsionally driving the screw into bone.

A method of assembling a tulip comprises the step of providing a tulip; and positioning a pedicle screw inside the tulip in pre-loaded condition. The method also includes the step of positioning the multi-segmented locking member in the groove or recess inside the tulip to pre-load the segment pieces in the unlocked condition. The method also includes the step of inserting the saddle into the tulip prior to tightening to hold the screw head and segmented locking member in place on assembly.

### BRIEF DESCRIPTION OF THE DRAWINGS

The invention will be described by way of example and with reference to the accompanying drawings in which:

FIG. 1 is a plan view of the pedicle screw assembly of a first embodiment of the invention prior to the tulip assembly connection of the pedicle screw.

FIG. 1A is an enlarged cross sectional view of an upper portion of the pedicle screw assembly after the tulip assembly is connected to the pedicle screw.

FIG. 1B is a cross sectional view of the assembly taken along lines B-B of FIG. 1A.

FIG. 1C is an alternative of the first embodiment showing a partial groove or recess and a two piece multi-segmented locking ring.

FIG. 2 is a plan view of FIG. 1 showing the pedicle screw assembled to the tulip moved upwardly inside the tulip as the locking ring engages.

FIG. 3 is an exploded cross sectional view of the assembly showing the tulip, the saddle, the multi-segmented locking ring and the pedicle screw.

FIG. 4 is an enlarged cross sectional view of the saddle.

FIG. 5 is an enlarged cross sectional view of the split ring.

FIG. 6 is an enlarged cross sectional view of an upper portion of the pedicle screw assembly after the tulip assembly is connected to the pedicle screw of the present invention wherein the lateral outer diameter or side of recess groove of the tulip is conically tapered.

FIG. 7 is an enlarged cross sectional view of an upper portion of the pedicle screw assembly after the tulip assembly is connected to the pedicle screw of the present invention wherein the lateral outer diameter or side of recess groove of the tulip is round forming a toroidal groove.

FIG. 8 is a section view of the assembly of a second alternative embodiment showing the locking segment pieces as a plurality of straight pieces.

FIG. 8A is a perspective view of the locking segment pieces of FIG. 8.

FIG. 8B is a refinement to the locking segment pieces of FIG. 8.

FIG. 9 is a section view of the assembly of a third alternative embodiment showing the locking segment pieces as a pair of “V” shaped pieces.

FIG. 9A is a perspective view of the pair of “V” shaped locking edges of FIG. 9.

FIG. 9B is a perspective view of the pair of “V” shaped locking segment pieces having contoured edges for holding the screw head.

### DETAILED DESCRIPTION OF THE INVENTION

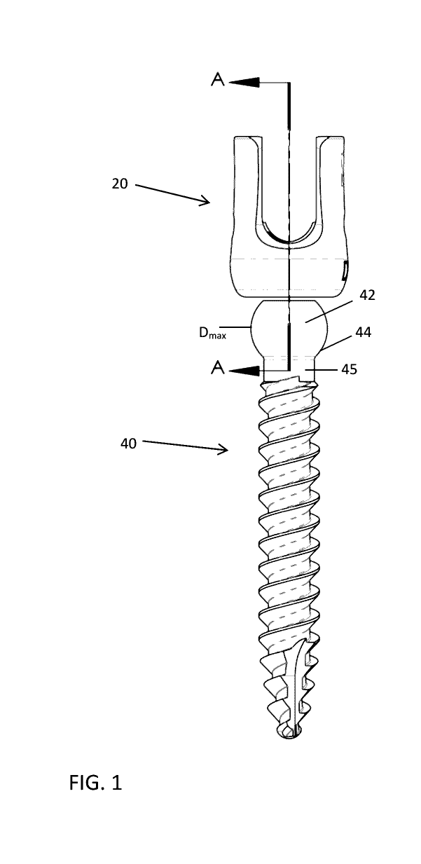

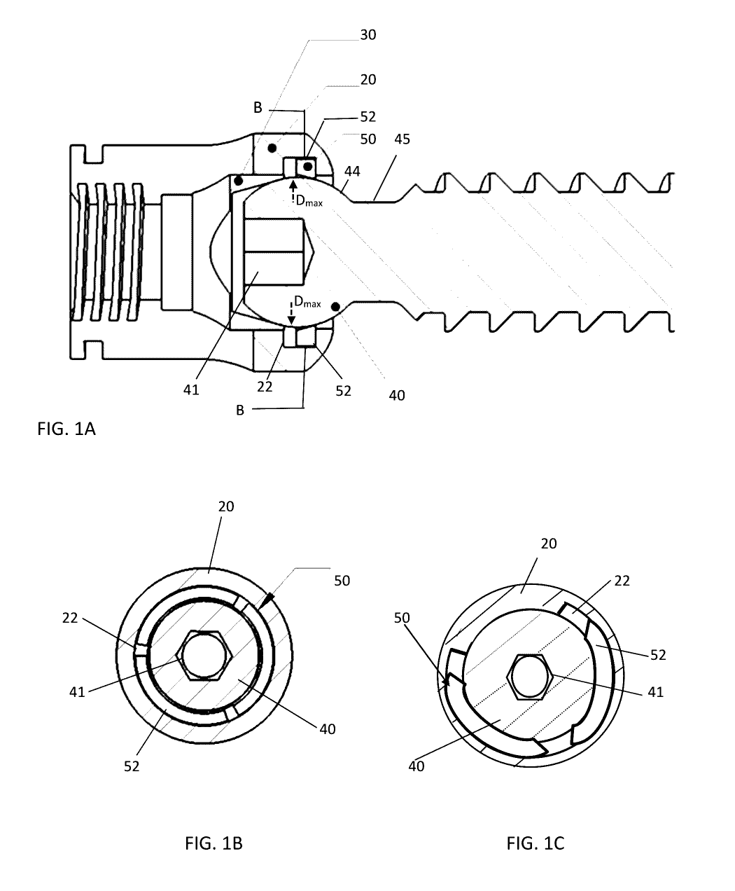

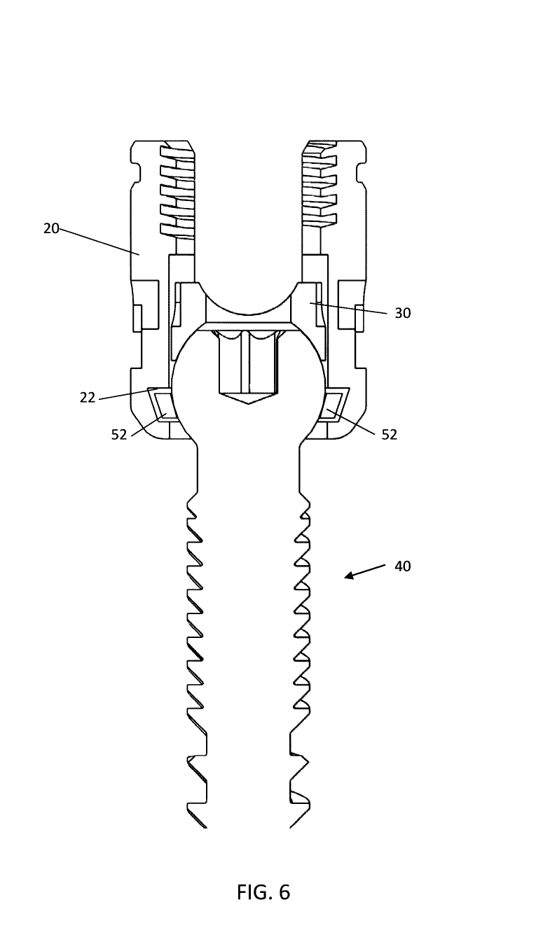

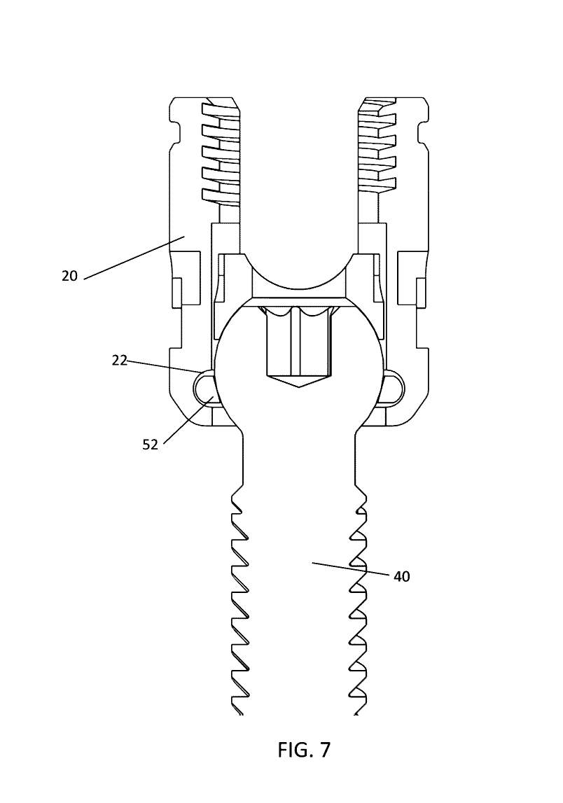

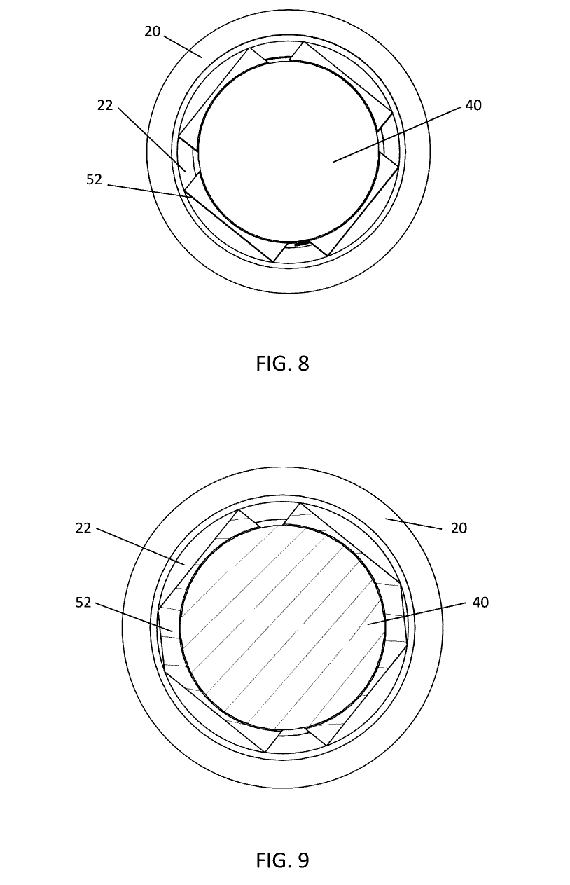

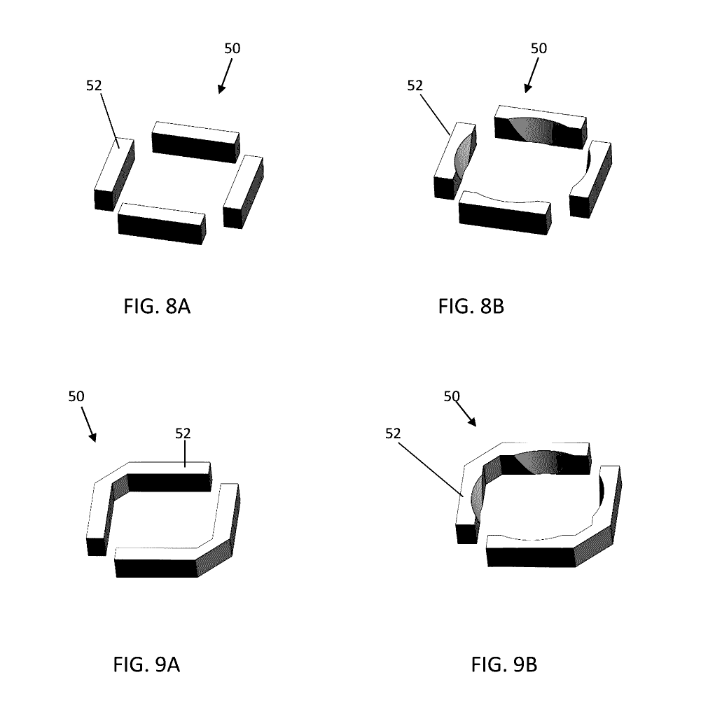

With reference to the drawings, various embodiments of the present invention are shown. In FIGS. 1-3. A first exemplary embodiment shows the multi-segment locking member 50 having arcuate segment pieces 52 held in a square recess or groove 22 of a tulip 20. In FIG. 1B, there are 3 segment pieces 52. In FIG. 1C, the groove 22 extends in a partial annular direction about 270 degrees and has two arcuate segment pieces 52. In FIG. 6, the tulip 20 has a conically tapered groove 22 and in FIG. 7 the recess or groove 22 is rounded. These are additional exemplary variations of the optional shapes of the recess or groove 22. In FIGS. 8, 8A and 8B, another second alternative embodiment is shown wherein the multi-segment locking member 50 can have straight segment pieces 52. In FIGS. 9, 9A and 9B, a third exemplary embodiment is shown wherein the multi-segment locking member is made of two “V” shaped segment pieces 52. In FIGS. 8B and 9B, the top edges are shown contoured to flushly contact the bottom of a pedicle screw head. In every case, these shape variations are identified by the same reference numeral recognizing the shape alternatives of the segment pieces 52 whether straight, “V” or arcuate pieces with or without contoured top edges or the recess or undercut groove 22 whether square, round or conically tapered full or partial all perform the same function and meet the same purpose of the invention despite alternative shapes. Accordingly, the following description of the multi-segment locking member 50 whether shaped as a ring or two opposing “V” shapes or a square sided four piece straight box, all achieve the claimed inventive concept which is described in detail as follows.

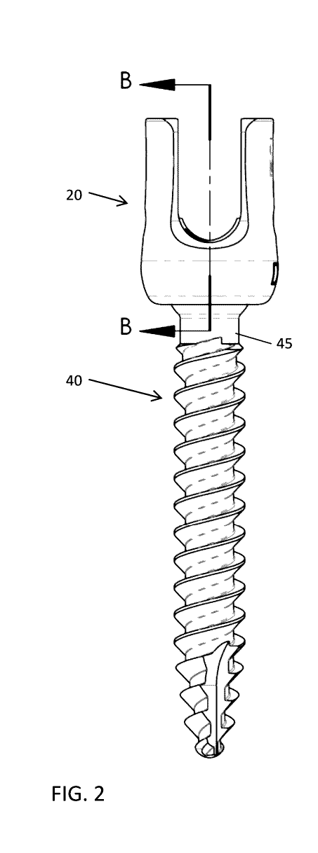

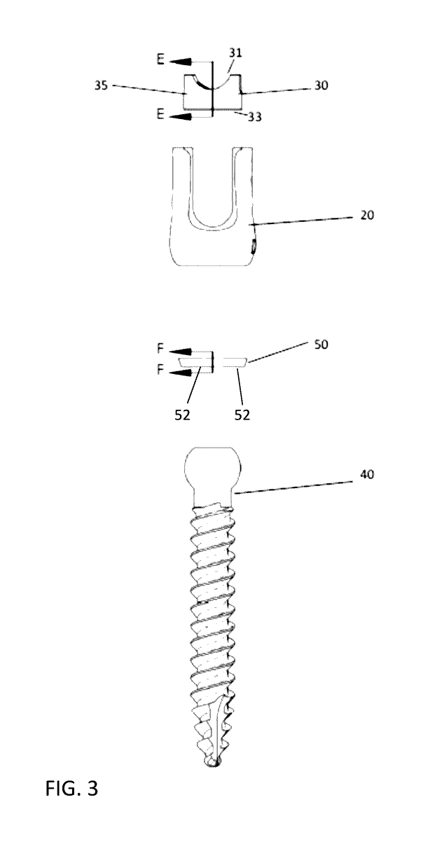

With reference to FIG. 3, an exploded view of a pedicle screw assembly 10 is illustrated. The pedicle screw assembly 10 has a bone screw 40, a tulip 20 and a multi-segmented locking member formed as a locking ring 50 having two or more segmented pieces 52 configured to be internal of the tulip positioned and held in a recess 22 of an inner surface of the tulip 20. The assembly 10 further includes a saddle 30, as shown in FIG. 4, having a proximal end 31 for engaging a rod and a distal end 33 for receiving the bone screw 40. The saddle 30 has an exterior surface 35 positioned between the ends. The outer surface 35 is sized to move axially inside the tulip 20. The saddle 30 holds the locking ring 50 in an initial pre-loaded position where it is held in an exterior recess or a groove 32 upon insertion of the bone screw 40 into the tulip, the saddle 30 moves proximally allowing the head 42 to move past the locking ring 50 allowing the locking ring 50 to be inserted in an unexpanded condition into the recess 22 thereafter locking the head 42 of the bone screw 40 into the tulip 20. Upon insertion of the saddle, the locking ring 50, is held against the head 42 of the bone screw 40 and cannot fall out.

With reference to FIGS. 1-1B, the assembly of the pedicle screw assembly 10 onto a pedicle screw 40 is illustrated. Initially, the tulip 20 with or without a saddle 30 has the bone screw installed through the distal opening of the tulip 20 and thereafter the segment pieces of the locking ring 50 are inserted and held inside the tulip 20 in a straight, round or conically inwardly tapered recess or groove 22, this is shown best in FIGS. 1A and 1B or 6 and 7 respectively. The locking ring 50 which is configured to fit into the recess or groove 22 of the tulip 20 which locks the head 42 and when inserted the saddle 30 pushes against the screw head 42 insures the multi-segmented locking ring 50 cannot fall out of the tulip 20.

In one preferred embodiment shown in FIG. 1B, the multi-segmented locking ring 50 can be a two-piece or three piece assembly extending just less than 360 degrees fitting in a recess or groove 22 extending 360 degrees.

In an alternative shown in FIG. 1C, the multi-segmented locking ring 50 can be a partial ring having two or more segments fitted into a partial annular recess or groove which extends 180 to 270 degrees. In this embodiment, the function of the partial segments acts the same, but one segment piece can be eliminated.

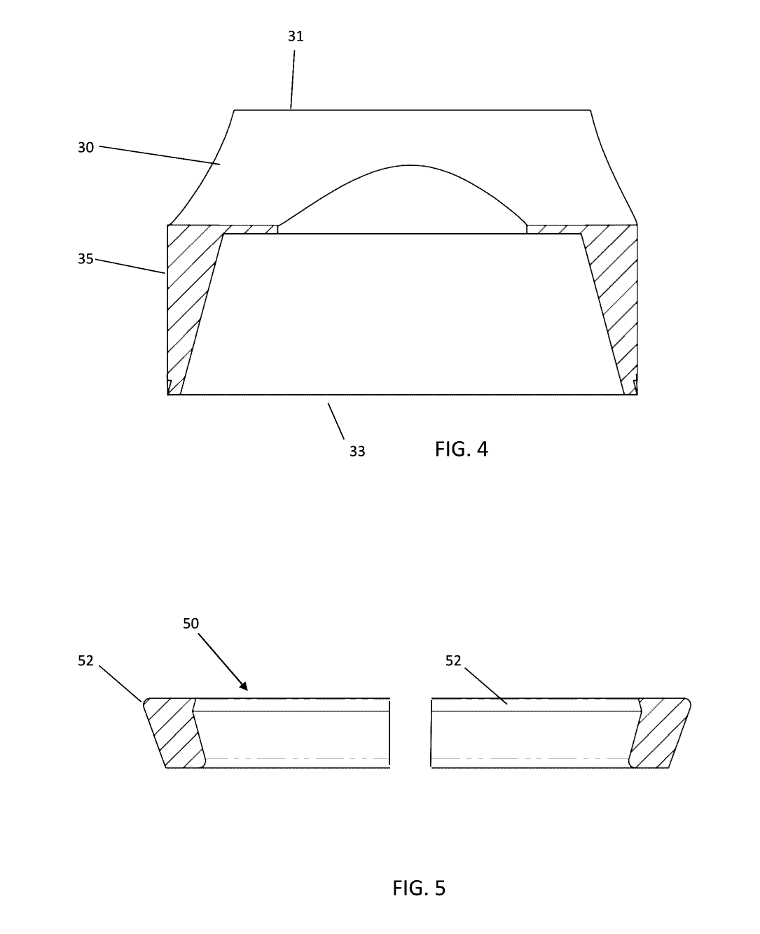

Accordingly, for this device to be manufactured, it is important that the locking ring or member 50 shown in FIG. 5 is assembled into a distal opening in the tulip 20. Inside the distal opening can be a tapered or conical recess 22 that tapers inwardly toward the distal end. Due to the size of the segmented pieces 52, it is important that on initial assembly, that the arcuate, straight or “V” shaped segment pieces 52 of the segmented member 50 are inserted into the distal end, if required, by being contracted slightly as it is pushed through an opening smaller than the size of the projected outer diameter of the member 50. Once the ring or member 50 is inside recess or groove 22 of the tulip 20, it can be held by the head 42. At this point, the saddle 30 can be positioned inside the tulip 20 and slid down to engage the top of the head 42. The ring or member 50 is then secured and will not dislodge without a tool or other device, not shown. Once this assembly of the locking ring or member 50 is fully secured by the saddle 30, the locking ring or member 50 is in an open unlocked position such that polyaxial movement of the pedicle screw 40 in the saddle 30 and tulip 20, as previously described, can occur until the set screw engages a rod and is tightened locking the screw head 42 against the saddle 30 bottom and the segment pieces 52 from movement. The locking ring or member 50 has segment pieces 52 preferably made of a high strength alloy having a very low creep percentage. This insures the stiffness at assembly is available to hold the screw head 42. The ring or member 50 is preferably made of titanium or a high strength stainless steel.

Accordingly, during manufacture, the tulip 20, saddle 30, bone screw 40 and locking ring 50 are pre-loaded as an assembly 10. As illustrated, the saddle 30 has an opening through which the pedicle screw 40 has a plurality of driving flats forming a hexagonal opening 41 that can be tightened or torqued down as required. This opening 41 can be formed by flats that are in a recess or can be any means for providing a torsional tightening of a pedicle screw into bone.

As illustrated, the tulip 20 has parallel walls that are opposing, the internal surface of the tulip 20 has threads for engaging a set screw. The tulip 20, as configured, is designed with a slotted opening for receiving a spinal rod or other similar mechanical device that can be positioned onto the upper surface of the saddle 30 which has a complimentary curved concavity for receiving a round or circular rod as illustrated. Once the set screw is put into position on top of the rod, the rod is then securely fastened against the saddle 30. Once this assembly is achieved, the polyaxial feature of the set screw is locked into position at the desired location.

These polyaxial features of the screw 40 are well understood in the art. It is important that the pedicle screw 40 have at least a partially hemispherical, spherical head 42 such that the lower head surface 44 is engaged by the tulip 20 and locking multi-segmented member 50 such that the screw 40 can be rotated in a polyaxial direction. This can be accomplished as indicated by hemispherical, a spherical head, a conical head or a radial array of loci or cylindrical surfaces or any other bulbous head configuration that is adapted to move in a polyaxial direction when inserted inside a tulip 20. Preferably, the screw 40 is made of an implantable metal such as stainless steel or, more preferably, titanium.

With reference again to FIG. 1A, the head 42 of the pedicle screw 40 has a maximum diameter Dₘₐₓ and this diameter decreases above and below this maximum Dₘₐₓ. Most particularly, in the exemplary screw 40, the lower portion 44 of the screw head 42 hemispherical external surface, decreases in size. When the screw 40 starts to move the saddle 30 with the locking member 50 affixed, the head 42 enters the saddle opening and prior to the member 50 being engaged, against the head 42 by the saddle 30 by abutting the tulip 20 internally, the maximum diameter Dₘₐₓ is shown above the inside diameter of the member 50. Accordingly, when the member 50 contracts against this lower portion 44 toward the screw shank 45. As this movement occurs, the tapered conical shaped recess 22 inside the tulip 20 comes into contact with the outer diameter of the locking ring or member 50 causing the tulip 20 to move slightly toward the screw shank 45, as shown.

The assembly being pre-loaded at the factory with the multi-segmented locking ring 50 pre-positioned with the saddle 30 and bone screw 40 all inside the tulip 20, allows the ring or member 50 to be substantially stronger than otherwise possible because the forces to engage the locking member 50 are far less than those required to stretch it over a pedicle screw head 42, additionally, this design allows much larger pedicle screws to be used in the tulip 20. At least one full size larger screw can be used, for example, a 7.5 mm pedicle screw in a normal tulip 20 can be replaced with an 8.5 mm size.

The exemplary screw 40 was an 8.5 mm screw. The invention can be of any suitable size for the required purpose allowing the tulip to be universally adaptable to any number of thread sized pedicle screws.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

1. A pedicle screw tulip assembly comprising:
a pedicle bone screw, the screw having a head and a threaded shank;
a tulip;
a multi-segmented locking member consisting of two, three or four separate segment pieces having circumferential gaps between the segments to form a segmented locking feature without any bending of the segments on insertion in the tulip, the multi-segmented locking member being internal of the tulip positioned in a recess or undercut groove of an inner surface of the tulip;
a saddle having a proximal end for engaging a rod and a distal end for receiving the head of the bone screw, the saddle having an exterior surface positioned between the ends, the outer surface being sized to move axially inside the tulip; and
wherein upon assembly of the pedicle screw into the tulip at least a maximum diameter of the head is positioned above the recess or undercut groove allowing the two or more separate segment pieces of the multi-segmented locking member to be inserted into the recess or groove thereby securing the pedicle screw inside the tulip.

2. The pedicle screw tulip assembly of claim 1 wherein the saddle, when placed on the screw head, further prevents the multi-segmented locking member from dislodging.

3. The pedicle screw tulip assembly of claim 1 wherein the recess or undercut groove of the tulip has a conical surface tapering inward distally.

4. The pedicle screw tulip assembly of claim 1 wherein a width of each of the segment pieces of the locking member is larger than a depth of the recess or groove of the tulip wherein a portion of the segment pieces overhang beyond the groove obstructing some of the distal opening.

5. The pedicle screw tulip assembly of claim 1 wherein the bone screw has one of the following head shapes; at least partially a hemispherical or spherical head, conical or a radial array or loci of cylindrical surfaces or any other bulbous head.

6. The pedicle screw tulip assembly of claim 1 wherein the head has a driving feature for torsionally driving the screw into bone.

7. A method of assembling a tulip comprises the step of:
providing a tulip; and
positioning a multi-segmented locking member inside the tulip in pre-loaded condition wherein the multi-segmented locking member consists of two, three or four separate segment pieces having circumferential gaps between the segments to form a segmented locking feature without any bending of the segments on insertion in the tulip, the multi-segmented locking member being internal of the tulip positioned in a recess or undercut groove of an inner surface of the tulip wherein upon assembly of the pedicle screw into the tulip at least a maximum diameter of the head is positioned above the recess or undercut groove allowing the two or more separate segment pieces of the multi-segmented locking member to be inserted into the recess or groove thereby securing the pedicle screw inside the tulip.

8. The method of assembling a tulip of claim 7 also includes the step of positioning a head of a pedicle screw in the tulip past a recess or groove of the tulip and placing the multi-segmented locking member into the groove or recess inside the tulip to pre-load the assembly.

9. The method of assembling a tulip of claim 8 also includes the step of inserting a saddle into the tulip to hold the head of the pedicle screw against the multi-segmented locking member.

10. A modular head pedicle screw assembly comprising:
a pedicle bone screw, the screw having a head and a threaded shank;
a tulip;
a multi-segmented locking ring consisting of two, three or four separate pieces of arcuate segments having circumferential gaps between the segments to form a segmented locking feature without any bending of the segments on insertion in the tulip, the multi-segmented locking ring being internal of the tulip positioned in a recess or undercut groove of an inner surface of the tulip;
a saddle having a proximal end for engaging a rod and a distal end for receiving the head of the bone screw, the saddle having an exterior surface positioned between the ends, the outer surface being sized to move axially inside the tulip; and
wherein upon assembly of the pedicle screw into the tulip at least a maximum diameter of the head is positioned above the recess or undercut groove allowing the two or more separate pieces of arcuate segments of the multi-segmented locking ring to be inserted into the recess or groove thereby securing the pedicle screw inside the tulip.

11. The modular head pedicle screw assembly of claim 10 wherein the saddle, when placed on the screw head, further prevents the multi-segmented locking ring from dislodging.

12. The modular head pedicle screw assembly of claim 10 wherein the recess or undercut groove of the tulip has a conical surface tapering inward distally.

13. The modular head pedicle screw assembly of claim 10 wherein an outer diameter of the locking ring is larger than a distal opening of the tulip.

14. The modular head pedicle screw assembly of claim 10 wherein the bone screw has one of the following head shapes; at least partially a hemispherical or spherical head, conical or a radial array or loci of cylindrical surfaces or any other bulbous head.

15. The modular head pedicle screw assembly of claim 10 wherein the head has a driving feature for torsionally driving the screw into bone.

16. A method of assembling a tulip comprises the step of:
providing a tulip; and
positioning a multi-segmented locking ring inside the tulip in pre-loaded condition wherein the multi-segmented locking ring consists of two, three or four separate segment pieces having circumferential gaps between the segments to form a segmented locking feature without any bending of the segments on insertion in the tulip, the multi-segmented locking ring being internal of the tulip positioned in a recess or undercut groove of an inner surface of the tulip wherein upon assembly of the pedicle screw into the tulip at least a maximum diameter of the head is positioned above the recess or undercut groove allowing the two or more separate segment pieces of the multi-segmented locking ring to be inserted into the recess or groove thereby securing the pedicle screw inside the tulip.

17. The method of assembling a tulip of claim 16 also includes the step of positioning a head of a pedicle screw in the tulip past a recess or groove of the tulip and placing the multi-segmented locking ring into the groove or recess inside the tulip to pre-load the assembly.

18. The method of assembling a tulip of claim 17 also includes the step of inserting a saddle into the tulip to hold the head of the pedicle screw against the multi-segmented locking ring.
